(19) United States
(12) Patent Application Publication
(10) Pub. No.: US 2022/0218727 A1
(43) Pub. Date: Jul. 14, 2022

(54) METHODS AND COMPOSITIONS OF SUPPORTING HUMAN HEALTH DURING SPACE TRAVEL

(71) Applicant: Intrinsic Medicine, Inc., Seattle, WA (US)

(72) Inventors: Jason Ferrone, San Diego, CA (US); Alexander Martinez, Des Moines, WA (US)

(21) Appl. No.: 17/573,084

(22) Filed: Jan. 11, 2022

Related U.S. Application Data
(60) Provisional application No. 63/136,578, filed on Jan. 12, 2021.

(51) Int. Cl. A61K 31/702; A23L 33/125

(52) U.S. Cl. CPC A61K 31/702; A23L 33/125

(57) ABSTRACT

Described are methods of treating a spaceflight-associated disease or condition, methods of reducing the effects of spaceflight or a low gravity environment, and methods of treating a subject in a low gravity environment. The methods comprise administration of a composition comprising one or more human milk oligosaccharides (HMOs).

### RELATED APPLICATION

This application claims the benefit of U.S. Provisional Application No. 63/136,578 filed Jan. 12, 2021. The entire contents of the above-referenced application are incorporated by reference herein.

### BACKGROUND OF THE INVENTION

Space travel is known to be associated with numerous negative health effects, including immune system effects, accelerated aging, muscle atrophy, bone loss, balance, cardiac atrophy and other cardiovascular effects, and increased susceptibility to infection and/or reactivation of latent infection (Crucian et al. (2018), Immune System Dysregulation During Spaceflight: Potential Countermeasures for Deep Space Exploration Missions, Front. Immunol. 9: 1437; Perhonen et al., Cardiac atrophy after bed rest and spaceflight. J Appl Physiol (1985). 2001 August;91(2):645-53. doi: 10.1152/japp1.2001.91.2.645. PMID: 11457776; https://phys.org/news/2020-10-human-heart-space-mathematical.html; Mermel (2013), Infection Prevention and Control During Prolonged Human Space Travel, Clinical Infectious Diseases 56(1): 123-130; the contents of each of which are expressly incorporated by reference herein). It is believed that some of the adverse effects are due to the effects of microgravity and/or exposure to space radiation.

It has been reported that NF-κB was elevated in astronauts after short-duration spaceflight (Zwart et al. (2010), Capacity of Omega-3 Fatty Acids or Eicosapentaenoic Acid to Counteract Weightlessness-Induced Bone Loss by Inhibiting NF-κB Activation: From Cells to Bed Rest to Astronauts, American Society for Bone and Mineral Research, 25(5): 1049-1057; the contents of which are expressly incorporated by reference herein). Specifically, Zwart showed that NF-κB p65 expression was increased by almost 500% in space shuttle crew members (Id.). NF-κB is a transcriptional activator that induces transcription factors that play a role in inflammation, muscle atrophy, and bone resorption (Id.). Zwart also provided evidence that the omega-3 fatty acid, eicosapentaenoic acid, can decrease NF-κB activation in modeled weightlessness and suggested that inhibition of this transcriptional activator can mitigate the spaceflight-associated effects on bone, muscle, and immune function (Id.).

Despite the recognition in the art of the numerous negative health effects associated with spaceflight, there remains a need in the art to mitigate or treat such effects.

### SUMMARY OF THE INVENTION

The invention includes methods of treating a spaceflight-associated disease or condition in a subject in need thereof comprising administering to said subject a composition comprising one or more human milk oligosaccharides (HMOs). The invention includes treating subjects suffering from a spaceflight-associated disease or condition as well as treating subjects at risk of a spaceflight-associated disease or condition. The invention includes administration of the composition comprising the one or more HMOs during spaceflight and/or prior to space travel. Non-limiting examples of spaceflight-associated diseases or conditions include dysbiosis, immune dysregulation, muscle wasting, metabolic disorders, cardiac disease, neurobehavioral abnormalities, irritable bowel syndrome, inflammatory bowel disease, osteoporosis, frailty, skin hypersensitivity, allergic reactions, viral infection, sarcopenia, and inflammaging.

The invention additionally includes methods of treating a subject in a low gravity environment comprising administering to said subject a composition comprising one or more human milk oligosaccharides. The subject in the low gravity environment can be suffering from low gravity environment associated dysbiosis, immune dysregulation, muscle wasting, metabolic disorders, cardiac or neurobehavioral abnormalities associated with such low gravity environment. Also encompassed is a method of reducing signs or symptoms of a subject in a low gravity environment comprising administering to said subject a composition comprising one or more human milk oligosaccharide, wherein said subject exhibits signs or symptoms of a disorder selected from irritable bowel disease, inflammatory bowel disease, osteoporosis, frailty, skin hypersensitivity, allergic reactions, viral infection, sarcopenia or inflammaging. A low gravity environment includes, for example, low planetary orbit (e.g., low Earth orbit, low Moon orbit, or low Mars orbit), an interplanetary voyage or inhabiting a planet or moon with gravity less than 1 G.

The invention further encompasses methods of reducing the effects of spaceflight or a low gravity environment in a subject at risk thereof or suffering therefrom comprising administering to said subject a composition comprising one or more human milk oligosaccharides (HMOs).

The composition comprises an effective amount of the one or more human milk oligosaccharides.

In certain aspects, the composition comprises one or more HMOs selected from lacto-N-tetraose (LNT), lacto-N-neotetraose (LNnT), lacto-N-hexaose (LNH), lacto-N-neohexaose (LNnH), 2′ fucosyllactose (2′FL), 3′fucosyllacose (3′FL), lacto-difucotetraose (LDFT), lacto-N-fucopenaose II/II (LNFP II/III), lactose-N-fucopentaose I (LNFP I), lacto-N-difuco-hexaose I (LNDFH I), lacto-N-difuco-hexaose II (LNDFH II), difucosyl-para-lacto-N-neohexaose (DFpLNnH), difucosyllacto-N-hexaose c (DFLNH c), 3′ sialyllactose (3′SL), 6′ sialyllactose (6′SL), LS-tetrasaccharide a (LSTa), LS-tetrasaccharide b (LST b), LS-tetrasaccharide c (LST c), 3′-sialyl-N-acetyllactosamine (3′ SLN), 6′-sialyl-N-acetyllactosamine (6′ SLN), or disialyllacto-N-tetraose (DSLNT), or a combination of any of thereof. In yet other aspects, the one or more HMOs is selected from 2′FL, 3′FL, 3′SL, 6′SL, LNT, or LNnT, or a combination of any of thereof.

In yet further aspects, the composition can comprise a mixture of two, three, four or five human milk oligosaccharides. Exemplary mixtures include: 2′FL and LNT; 2′FL and LNnT; 2′FL, 3′FL, 3′SL, 6′SL and LNT; 3′SL and 6′SL; and 6′SL and LNT.

### DETAILED DESCRIPTION OF THE INVENTION

As used herein and in the appended claims, the singular forms “a,” “an,” and “the” include plural referents unless the context clearly dictates otherwise. Thus, for example, reference to “an oligosaccharide” includes a plurality of such oligosaccharides and reference to “the therapeutic agent” includes reference to one or more therapeutic agents and equivalents thereof known to those skilled in the art, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs.

Although many methods and reagents are similar or equivalent to those described herein, the exemplary methods and materials are disclosed herein. All publications mentioned herein are incorporated herein by reference in full for the purpose of describing and disclosing the methodologies, which might be used in connection with the description herein. Moreover, for terms expressly defined in this disclosure, the definition of the term as expressly provided in this disclosure will control in all respects, even if the term has been given a different meaning in a publication, dictionary, treatise, and the like.

The term “about” as used herein, in reference to a numerical value or range, allows for a degree of variability in the value or range, for example, within 10%, within 5%, or within 4%, or within 2% of the value or range.

The term “pharmaceutically acceptable carrier,” “pharmaceutically acceptable excipient,” “physiologically acceptable carrier,” or “physiologically acceptable excipient” as used herein, refers to a pharmaceutically-acceptable material, composition, or vehicle, such as a liquid or solid filler, diluent, excipient, solvent, or encapsulating material. Each component must be “pharmaceutically acceptable” in the sense of being compatible with the other ingredients of a pharmaceutical formulation. It must also be suitable for use in contact with the tissue or organ of humans and animals without excessive toxicity, irritation, allergic response, immunogenecity, or other problems or complications, commensurate with a reasonable benefit/risk ratio. Examples of “pharmaceutically acceptable carriers” and “pharmaceutically acceptable excipients” can be found in the following, Remington: The Science and Practice of Pharmacy, 21st Edition; Lippincott Williams & Wilkins: Philadelphia, Pa., 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, Fla., 2004.

The term “spaceflight” means travel outside of the Earth's atmosphere, for example on the Space Shuttle, the International Space Station, a satellite, a rocket, or other space vehicle, such that microgravity conditions exist. Spaceflight includes travel in Earth's orbit, as well as travel through space, such as between planets.

“Microgravity” is a state in which there is very little net gravitational force, for example, gravity less than about 1 G. Microgravity conditions exist in space, for example, aboard the Space Shuttle, the International Space Station, a satellite, or a rocket while in flight outside the Earth's atmosphere. Simulated microgravity is microgravity which is simulated by a set of Earth-based conditions that mimic microgravity, such as by balancing gravity with equal and opposite forces (for example, shear force, centripetal force, Coriolis forces, buoyancy, and/or magnetic field). In one example, simulated microgravity may be generated by use of a clinostat, such as a rotating wall vessel (RWV). In another example, simulated microgravity may be generated by a random positioning machine (RPM). The terms “microgravity conditions” and “microgravity” are used synonymously herein. Normal gravity is the gravity normally experienced on Earth, such as on the surface of the Earth and/or in its atmosphere (for example, in aircraft in the atmosphere of the Earth). Gravity is measured in terms of acceleration due to gravity, denoted by g. The strength (or apparent strength) of Earth's gravity varies with latitude, altitude, local topography, and geology. In some examples, normal gravity (such as 1G) is about 9-10 m/s², for example, about 9.7-9.9 m/s². In particular preferred embodiments, normal gravity is that experienced on the surface of the Earth under normal gravity at that location on the Earth. The terms “microgravity” and “low gravity” are used interchangeably herein.

A “low gravity environment” is an environment of low gravity or microgravity as described herein. Examples of a low gravity environment include a low planetary orbit (e.g., low Earth orbit, low Moon orbit or low Mars orbit), an interplanetary voyage or inhabiting a planet or moon with gravity less than 1 G.

The terms “spaceflight associated disease or condition” and “spaceflight induced disease or condition,” and the like include diseases, disorders and conditions that occur during spaceflight or in a low gravity environment including, but not limited to, those associated with the effects of microgravity (or low gravity) and/or spaceflight associated radiation exposure. Non-limiting examples of such conditions include spaceflight-induced immune dysregulation and associated comorbidities, spaceflight-induced muscle atrophy, spaceflight orthostatic intolerance, spaceflight or microgravity-associated bone loss, as well as dysbiosis, irritable bowel syndrome, inflammatory bowel disease, immune dysregulation, osteoporosis, frailty, skin hypersensitivity, allergic reactions, viral infection, sarcopenia, inflammaging, muscle wasting, metabolic disorders, cardiac atrophy or neurobehavioral abnormalities associated with a low gravity environment or spaceflight.

The term “subject” as used herein, refers to an animal, including, but not limited to, a primate (e.g., human, monkey, chimpanzee, gorilla, and the like), rodents (e.g., rats, mice, gerbils, hamsters, ferrets, and the like), lagomorphs, swine (e.g., pig, miniature pig), equine, canine, feline, and the like. The terms “subject” and “patient” are used interchangeably herein. For example, a mammalian subject can refer to a human patient. In preferred aspects, the subject is a human patient. The subject can, for example, be an astronaut.

The term “release controlling excipient” as used herein, refers to an excipient whose primary function is to modify the duration or place of release of the active substance from a dosage form as compared with a conventional immediate release dosage form. The term “non-release controlling excipient” as used herein, refers to an excipient whose primary function do not include modifying the duration or place of release of the active substance from a dosage form as compared with a conventional immediate release dosage form.

The term “substantially pure” as used herein in reference to a given oligosaccharide means that the oligosaccharide is substantially free from other biological macromolecules. The substantially pure oligosaccharide is at least 75% (e.g., at least 80, 85, 95, or 99%) pure by dry weight. Purity can be measured by any appropriate standard method, for example, by column chromatography, polyacrylamide gel electrophoresis, or HPLC analysis.

The terms “treat”, “treating” and “treatment,” as used herein, refers to ameliorating symptoms associated with a disease, condition, or disorder (e.g., a disease, condition or disorder associated with spaceflight or low gravity environment), including inhibiting the progress of the disease or disorder (e.g., a disease, condition or disorder associated with spaceflight or low gravity environment), reducing the severity of the disease or disorder (e.g., a disease, condition or disorder associated with spaceflight or low gravity environment), preventing or delaying the onset of the disease, condition or disorder symptoms, and/or lessening the severity or frequency of symptoms of the disease, condition, or disorder.

The present invention contemplates co-administration of the composition comprising the one or more human milk oligosaccharide and an additional active agent. As used herein “co-administration” means administration of at least two therapeutically active drugs or compositions (e.g., administration of the human milk oligosaccharide and an additional active agent, such as an additional therapeutic agent or nutritional supplement, or pharmaceutical compositions thereof), at different times or simultaneously or substantially simultaneously in either separate formulation or the same formulation/composition. When the at least two therapeutic agents are administered at different times, their administration can be separated by minutes, hours, days, weeks, or months, and/or be administered as part of the same treatment regimen.

An “effective amount” or a “therapeutically effective amount” of an agent (e.g., one or more HMOs or additional active agent) as described herein refers to an amount the active agent, alone or in combination with another active agent, that is sufficient to achieve a specific effect or result, and/or treats the disease or condition and/or the symptoms therefore, for example, alleviating, in whole or in part, symptoms associated with the disorder or condition, or halts or slows further progression or worsening of those symptoms, or prevents or provides prophylaxis for the disorder or condition. For example, an “effective amount” of a human milk oligosaccharide, as described herein, encompasses an amount that, alone or in combination with another human milk oligosaccharide, is effective to treat a spaceflight or low gravity associated disease, condition, or disorder.

The terms “active agent,” “drug,” “therapeutic agent,” are used interchangeably herein and refer to an agent administered as part of a method of treatment, alone or in combination with one or more pharmaceutically acceptable excipients and/or carriers, to a subject for treating, preventing, or ameliorating one or more symptoms of a disorder. The terms “active agent,” “therapeutic agent,” and “drug” as used herein includes, but are not limited to, human milk oligosaccharides.

The term “disorder” as used herein is intended to be generally synonymous, and is used interchangeably with, the terms “disease,” “syndrome” and “condition” (as in medical condition), in that all reflect an abnormal condition of the body or of one of its parts that impairs normal functioning and is typically manifested by distinguishing signs and symptoms.

The invention encompasses methods of treating a methods of treating a spaceflight-associated disease or condition in a subject in need thereof and also methods of treating a subject in a low gravity environment, comprising administering to said subject a composition comprising one or more human milk oligosaccharide; for example, such treatment prevents or slows the progression of dysbiosis, immune dysregulation, muscle wasting, metabolic disorders, cardiac atrophy, neurobehavioral abnormalities, irritable bowel syndrome, inflammatory bowel disease, osteoporosis, frailty, skin hypersensitivity, allergic reactions, viral infection, sarcopenia or inflammaging associated with such low gravity environment. The subject can be a subject exhibiting a sign or symptom of a spaceflight-associated disease or a condition or disorder associated with a low gravity environment. The composition comprises an effective amount of one more oligosaccharides. Alternatively, the subject can be at risk of developing a spaceflight-associated disease or a condition or disorder associated with a low gravity environment. In specific examples, the HMOs are administered in amount from about 1 to about 15 g (total HMOs) once or twice a day for one or more days, for one or more weeks, for one or more months, and/or for the duration of the spaceflight. In another example, the HMOs are administered in an amount from about 2.6 to about 7.5 g twice a day, once or twice a day for one or more days, for one or more weeks, for one or more months, and/or for the duration of the spaceflight. A subject at risk for developing a spaceflight-associated disease or condition or disorder associated with a low gravity environment includes a subject that is planning to travel to space and/or a low gravity environment and/or a subject that is in spaceflight and/or a low gravity environment and is not yet exhibiting a sign or symptom of a spaceflight-associated disease or condition or disorder associated with a low gravity environment. For example, a subject can be treated with the composition before spaceflight, for example, 2-4 weeks pre-flight. In one example, the HMOs are administered in an amount from about 1 to about 15 g (total HMOs) once or twice a day one or more days pre-flight; e.g., 2-4 weeks pre-flight. In another example, the HMOs are administered in amount from about 2.6 to about 7.5 g twice a day, in a period of time pre-flight; e.g., 2-4 weeks pre-flight.

As described above, examples of conditions that can be treated or prevented according to the method described herein include spaceflight-induced immune dysregulation and associated comorbidities, spaceflight-induced muscle atrophy, spaceflight orthostatic intolerance, spaceflight or microgravity-associated bone loss, as well as dysbiosis, irritable bowel syndrome, inflammatory bowel disease, immune dysregulation, osteoporosis, frailty, skin hypersensitivity, allergic reactions, viral infection, sarcopenia, inflammaging, muscle wasting, metabolic disorders, cardiac atrophy or neurobehavioral abnormalities associated with a low gravity environment or spaceflight. In certain aspects, the subjected treated with the composition is a subject that has exhibited a signs or symptom of one or more of the following disorders while in said low gravity environment: dysbiosis, immune dysregulation, muscle wasting, metabolic disorders, cardiac atrophy or neurobehavioral abnormalities associated with such low gravity environment.

Without wishing to be bound by theory, the effects of HMOs on spaceflight-induced disease and disorders and conditions and disorders associated with a low gravity environment may be at least partially mediated by inhibition of NF-kB signaling. Kang et al. (2018) (3′-Sialyllactose as an inhibitor of p65 phosphorylation ameliorates the progression of experimental rheumatoid arthritis, British Journal of Pharmacology 175(23): 4295-4309) showed that 3′SL inhibited phosphorylation of p65, part of the NF-kB signaling pathway, in a murine rheumatoid arthritis model.

In certain aspects, the disease or condition treated is spaceflight-induced dysbiosis or dysbiosis associated with low gravity environment. Dysbiosis is defined by the loss or gain of bacteria that promote health or disease (see, for example, Wilkins et al. (2019), Defining Dysbiosis for a Cluster of Chronic Diseases, Nature Briefing 9: 12918; the contents of which are expressly incorporated by reference herein). The subject can, for example, be exhibiting a sign or symptom of dysbiosis wherein the dysbiosis is determined by fecal microbiome analysis such as 16S ribosomal RNA sequencing. For example, if the fecal microbiome analysis shows that that the subject's fecal microbiome contains less Bifidobacterium and Faecalibacterium (e.g., F. prausnitzii) compared to the subject's microbiome while on Earth, then the subject is treated with the composition comprising the one or more human milk oligosaccharides as described herein. In yet other aspects, the subject has an increased level of salivary cortisol, plasma IL-6, plasma IL-1β, plasma TNF-α, plasma c-reactive protein, MCP-1, MIP1-α, fecal calprotectin, compared to levels thereof in said subject on Earth.

That the spaceflight impacts the microbiome and specifically that the microbial constitution of the gastrointestinal tract, skin, nose and tongue changes during space travel has been demonstrated by Voorhies et al. (2019), Study of the impact of long-duration space missions at the International Space Station on the astronaut microbiome, Sci Rep 9, 9911 (2019). Voorhies suggested that changes to skin microbiome might be a factor in the increased incidence of skin reactions and hypersensitivity experienced by astronauts. Spaceflight-induced immune dysregulation and associated comorbidities, and inflammaging include, but are not limited to, autoimmunity (e.g., urticaria, skin rash) as well as allergies/hypersensitivity, inflammatory conditions including chronic inflammatory conditions. Biomarkers of such immune regulations which can be measured before and during treatment include, but are not limited to, IL-6, CRP, IL-17, and ESR. Such conditions are described, for example, in Smith, J. K. IL-6 and the dysregulation of immune, bone, muscle, and metabolic homeostasis during spaceflight. npj Microgravity 4, 24 (2018) and Bucheim et al. (2019), Stress Related Shift Toward Inflammaging in Cosmonauts After Long-Duration Space Flight, Front. Physiol., 19; the contents of each of which are expressly incorporated by reference herein.

The condition can also be a neurobehavioral abnormality associated with spaceflight and/or low gravity environment or associated with dysbiosis (see, for example, Maguire et al. (2018), Gut dysbiosis, leaky gut, and intestinal epithelial proliferation in neurological disorders: towards the development of a new therapeutic using amino acids, prebiotics, probiotics, and postbiotics, Reviews in the Neurosciences, 30(2); the contents of which are expressly incorporated by reference herein). Spaceflight is associated with risk factors that negatively impact neurobehavioral function, including cognition (Nasrini et al. (2020), Cognitive Performance During Confinement and Sleep Restriction in NASA's Human Exploration Research Analog (HERA), Front. Physiol., 28 Apr. 2020; the contents of which are expressly incorporated by reference herein). The neurobehavioral abnormality can be an anxiety disorder, depression, brain fog, stress, and negatively impacted cognitive performance. The method can entail administering a neurobehavioral test to a subject in a low gravity environment or space and determining if neurobehavioral function is impacted and the subject is in need to treatment. Alternatively or in addition, the method comprises using a neurobehavioral test to monitor the effectiveness of the treatment. An exemplary neurobehavioral test battery is described by Nasrini et al. (2020).

The condition treated according to the methods described herein can also be spaceflight induced or low gravity environment associated bone and/or muscle loss. It is well-recognized that in the microgravity environment of spaceflight, bone and muscle atrophy occurs. In order to mitigate the effects of microgravity on bone and/or muscle loss, astronauts are recommended or “prescribed” multiple hours of exercise per day (see, e.g., https://www. nasa. gov/mission_pages/station/research/station-science-101/bone-muscle-loss-in-microgravity/; the contents of which are expressly incorporated by reference herein). The composition described herein can be administered to the subject to treat, prevent or mitigate spaceflight induced or low gravity environment associated bone and/or muscle loss. The composition can be administered to the subject in combination with the “prescription” of exercise.

The condition can also be spaceflight induced or low gravity environment associated cardiac atrophy or cardiovascular deconditioning. Such cardiac atrophy and cardiovascular deconditioning has been described, for example, in Perhonen et al. (1985), Cardiac atrophy after bed rest and spaceflight, J Appl Physiol 91(2): 645-53 and https://phys.org/news/2020-10-human-heart-space-mathematical.html; the contents of each of which are expressly incorporated by reference herein.

Spaceflight and low gravity environments are associated with increased susceptibility to infection and/or reactivation of latent infection (Mermel (2013), Infection Prevention and Control During Prolonged Human Space Travel, Clinical Infectious Diseases 56(1): 123-130; the contents of which are expressly incorporated by reference herein. Such infections also include, but are not limited to, respiratory or lung infections, skin infections, and urinary tract infections. Such infections include, for example, Salmonella, Shigella, Vibrio cholerae, E. coli, Polioviruses, Rotavirus and Respiratory Syncytial virus (RSV), Epstein Bar virus (EBV), Varicella Zoster virus (VZV), and influenza infections. Although not wishing to be bound by theory, the HMOs may reduce susceptibility to infection (including new and latent reinfection) through anti-adhesive effects. (Facinelli et al. (2018) (Breast milk oligosaccharides: effects of 2′-fucosyllactose and 6′-sialyllactose on the adhesion of Escherichia coli and Salmonella fyris to Caco-2 cells, The Journal of Maternal-Fetal & Neonatal Medicine, 32(17): 2950-2952) reported a reduction of E. Coli adhesion to Caco-2 cells was observed in the presence 2′FL and 6′ SL. The HMOs may additionally or alternatively improve maintenance of the epithelial barrier, for example, by maintenance of the tight junction proteins and/or by supporting healthy mucin via short-chain fatty acids (SCFA) production to prevent leaky gut.

An “oligosaccharide” is a saccharide polymer containing a small number (typically three to ten) of simple sugars (monosaccharides). A “human milk oligosaccharide” is an oligosaccharide found in human milk. As used herein, the term “human milk oligosaccharide” includes natural or native oligosaccharides found in human milk, as well as pharmaceutically acceptable salts, derivatives, prodrugs, and solvates thereof. The term “natural human milk oligosaccharide” or “natural HMO” refers to human milk oligosaccharides naturally found in human milk. Natural human milk oligosaccharides (HMOs) are separated into different classes including, for example, sialylated human milk oligosaccharides and fucosylated oligosaccharides. HMOs include natural sialylated human milk oligosaccharides and fucosylated oligosaccharides, as well as non-naturally occurring derivatives thereof.

Sialyllactose is a class of human milk oligosaccharides (HMOs) that appear in two different forms in human milk. These two forms are 3′-sialyllactose (3′-SL) and 6′-sialyllactose (6′-SL):

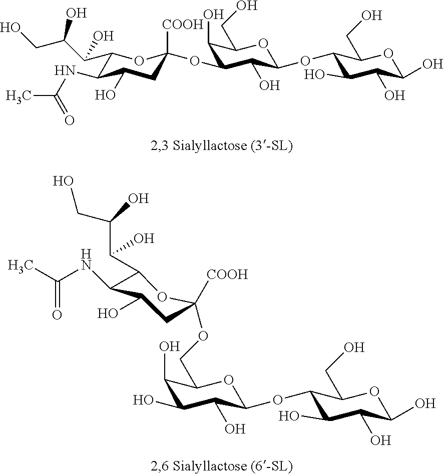

The terms “3′-SL” and “3′SL” are used interchangeably herein. Similarly, the terms “6′-SL” and “6′SL” are used interchangeably herein. Sialyllactoses have been shown to modulate acute and chronic immune responses in both murine and human derived macrophages stimulated with LPS and various pro-inflammatory cytokines. Both 3′SL and 6′SL have shown reductions in interleukin (IL)-1β, IL-2, IL-4, IL-6, IL-12, interferon (IFN) γ or TNF-α in vitro, with 3′-SL exhibiting more significant reductions. In addition, 3′SL has been shown to reduce other key target proteins, including PDL1, COX2 and select chemokines, such as CCL2 (also known as monocyte chemoattractant protein 1 (MCP1)) and CCL5. In vivo data in mouse models of rheumatoid arthritis, which include an LPS challenge, sialyllactose has shown benefit in clinical assessments of disease when administered orally.

Fucosylated oligosaccharides are a class of human milk oligosaccharides (HMOs) that have been associated with the production of anti-inflammatory short-chain fatty acids. Fucosylated oligosaccharides include, for example, 2′-fucosyllactose, 3-fucosyllactose, difucosyllactose, lacto-N-fucopentaoses (that is to say lacto-N-fucopentaose I, lacto-N-fucopentaose II, lacto-N-fucopentaose III and lacto-N-fucopentaose V), lacto-N-difucohexaose I, fucosyllacto-N-hexaose, Difucosyllacto-N-hexaose I and Difucosyllacto-N-neohexaose II. In certain aspects, the fucosylated non-digestible oligosaccharide is 2′-fucosyllactose (2′-FL). In certain aspects, the fucosylated oligosaccharide is 2′-fucosyllactose (2′-FL), 3-fucosyllactose (3′-FL), difucosyllactose (DFL). In yet further aspects, the fucosylated oligosaccharide is 2′-FL. As used herein, a “fucosylated oligosaccharide” is an oligosaccharide having the three sugar units backbone, wherein each of the sugar units (fucose (Fuc), galactose (Gal), and glucose (Glc)) can be independently either in its native form or in a modified form. For example, the modified form of a sugar unit can be a sugar unit, in which at least one or more (e.g., 1, 2, 3, or more) of the hydroxyl groups is replaced with hydrogen, alkyl or a functional group; such as, for example, hydrogen, substituted or unsubstituted C1-C6 alkyl (e.g., methyl, ethyl), or substituted or unsubstituted amine group.

Fucosyllactose (FL) is a fucosylated non-digestible oligosaccharide present in human milk but not in cow milk. The primary fucosylated HMO is 2′-fuscosyllactose or 2′FL. It consists of three monosaccharide units, fucose, galactose and glucose linked together. Lactose is a galactose unit linked to a glucose unit via a beta 1,4 linkage. A fucose unit is linked to a galactose unit of a lactose molecule via an alpha 1,2 linkage (2′-fucosyllactose, 2′-FL) or via an alpha 1,3 linkage to the glucose unit of a lactose (3-Fucosyllactose, 3-FL). 2-'FL has the chemical structure shown below:

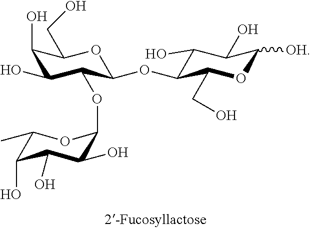

The terms 2′-fucosyllactose or “2′-FL” and “2′FL” are used interchangeably herein. 2′-fucosyllactose has been granted generally regarded as safe (GRAS) status in the U.S. and is regarded by the Europe Food Safety Authority as safe for infant and follow-on formula. 2′-FL has been shown to have many beneficial properties, such as improving of gut health through modulation of the gut microbiome as well as reduction of local gut inflammation in models of necrotizing enterocolitis and other inflammatory bowel diseases. In addition, 2′-FL has been shown to have positive effects on gut epithelial barrier function and also independent anti-inflammatory effects through the reduction in TNFα and IL-8.

Derivatives of natural HMOs can be chemically modified as compared to the natural HMO. In certain aspects, the derivative of the natural HMO retains at least 50%, at least 60%, at least 70% or more (including, e.g., at least 80%, at least 90%, at least 95%, at least 98%, at least 99% and up to 100%) of the biological functions of a natural HMO. Such biological effects include, but are not limited to anti-inflammatory effects, anti-bacterial adhesion effects, prebiotic effects and/or or effects in treating or preventing the spaceflight-associated disease or condition and/or the disease or condition associated with a low gravity environment.

HMOs include, but are not limited to, compounds having a structure of Formula I, I(a), II, or IIIa:

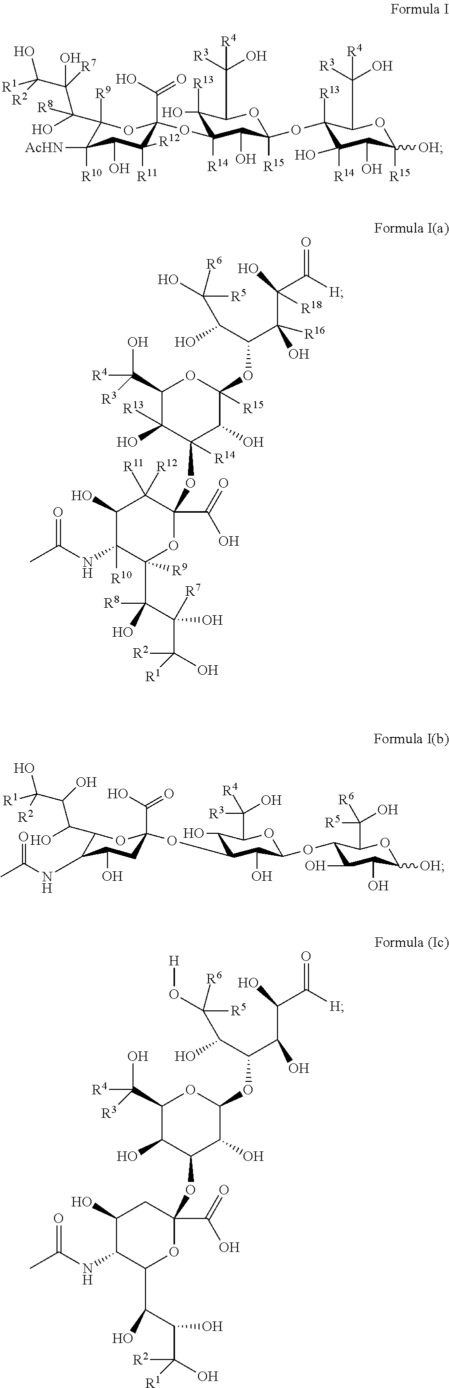

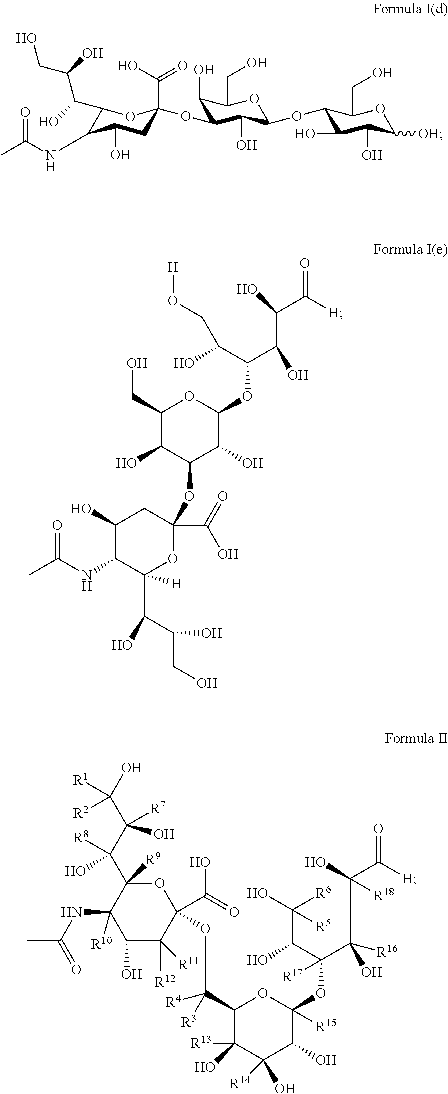

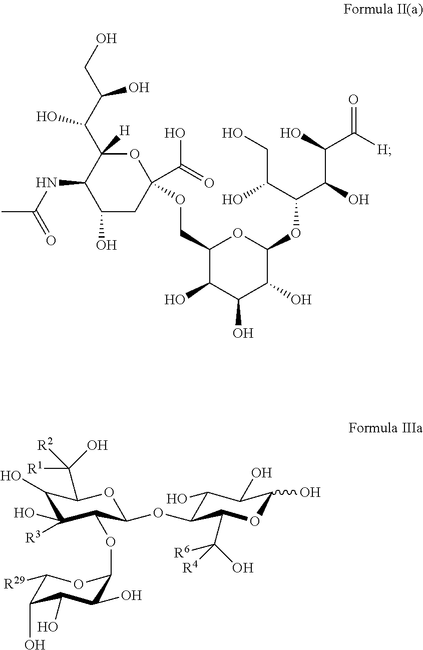

or a pharmaceutically acceptable salt, solvate, or prodrug thereof, wherein:

R¹-R¹⁸ are each independently selected from H, D, a halo, an unsubstituted or substituted (C₁-C₆)alkyl, an unsubstituted or substituted (C₁-C₆)heteroalkyl, an unsubstituted or substituted (C₂-C₆)alkenyl, an unsubstituted or substituted (C₂-C₆)heteroalkenyl, an unsubstituted or substituted (C₃-C₆)alkynyl, an unsubstituted or substituted (C₃-C₆)heteroalkynyl, an unsubstituted or substituted (C₄-C₈)cycloalkyl, an unsubstituted or substituted heterocycle, an unsubstituted or substituted aryl, —ROR′, —RN(R′)₂, —RSSR′, —SH, —RSOR′, —RSO₂R′, —RSO₂H, —RSO₃H, —RC(═S)—R′, —ROH, —RC(═O)R′, —RNO₂, —RSR′, —RCN, —RNC, —RNNR′, —RC(═O)OR′, —ROC(═O)R′, —RC(═O)H, —RC(═O)OH, —RC(═O)N(R′)₂, —RN₃, —ROCN, —RNCO, —RONO₂, —RNO, —ROP(═O)(OH)₂, and —RB(OH)₂;

R is absent or a (C₁-C₅)alkyl;

R′ is independently selected from H, D, an unsubstituted or substituted (C₁-C₆) alkyl, an unsubstituted or substituted (C₁-C₆)heteroalkyl, an unsubstituted or substituted (C₂-C₆) alkenyl, an unsubstituted or substituted (C₂-C₆)heteroalkenyl, an unsubstituted or substituted (C₃-C₆)alkynyl, an unsubstituted or substituted (C₃-C₆)heteroalkynyl, an unsubstituted or substituted (C₄-C₈)cycloalkyl, an unsubstituted or substituted heterocycle, and an unsubstituted or substituted aryl; and

R²⁹ is an unsubstituted or substituted (C₁-C₆)alkyl.

In yet additional aspects, the HMO has the Formula IIIb:

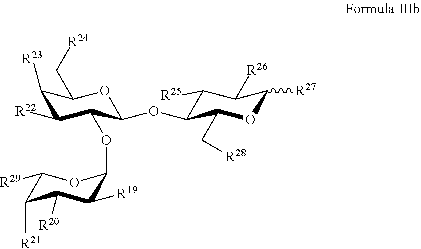

wherein:

one, two or three of R¹⁹-R²⁸ are each independently selected from the group consisting of hydrogen, an unsubstituted or substituted C₁-C₆ alkyl (including, but not limited to, methyl and ethyl) and N(R′)2 (wherein R′ is as defined above), the remainder or R₁₉-R₂₈ are —OH, and R²⁹ is substituted or unsubstituted C₁-C⁶ alkyl; or

one, two or three of R¹⁹-R²⁹ are each independently selected from NHC(O)R″, wherein R″ is unsubstituted or substituted (C₁-C₆) alkyl (including, but not limited to, methyl), the remainder or R₁₉-R₂₈ are —OH, and R²⁹ is substituted or unsubstituted C₁-C⁶ alkyl. In certain aspects, R²⁶ is NHC(O)CH₃ and R¹⁹-R²⁵ and R²⁷-R²⁸ are —OH, and R²⁹ is methyl.

The term “alkyl” refers to an organic group that is comprised of carbon and hydrogen atoms that contains single covalent bonds between carbons. Typically, an “alkyl” as used in this disclosure, refers to an organic group that contains 1, 2, 3, 4, 5, 6, 7, 8, 9, 10, 11, 12, 13, 14, 15, 16, 17, 18, 19, 20, or 30 carbon atoms, or any range of carbon atoms between or including any two of the foregoing values. Where if there is more than 1 carbon, the carbons may be connected in a linear manner, or alternatively if there are more than 2 carbons then the carbons may also be linked in a branched fashion so that the parent chain contains one or more secondary, tertiary, or quaternary carbons. An alkyl may be substituted or unsubstituted, unless stated otherwise.

The term “alkenyl”, refers to an organic group that is comprised of carbon and hydrogen atoms that contains at least one double covalent bond between two carbons. Typically, an “alkenyl” as used in this disclosure, refers to organic group that contains 2, 3, 4, 5, 6, 7, 8, 9, 10, 11, 12, 13, 14, 15, 16, 17, 18, 19, 20, or 30 carbon atoms, or any range of carbon atoms between or including any two of the foregoing values. While a C2-alkenyl can form a double bond to a carbon of a parent chain, an alkenyl group of three or more carbons can contain more than one double bond. In certain instances, the alkenyl group will be conjugated, in other cases an alkenyl group will not be conjugated, and yet other cases the alkenyl group may have stretches of conjugation and stretches of non-conjugation. Additionally, if there is more than 2 carbon, the carbons may be connected in a linear manner, or alternatively if there are more than 3 carbons then the carbons may also be linked in a branched fashion so that the parent chain contains one or more secondary, tertiary, or quaternary carbons. An alkenyl may be substituted or unsubstituted, unless stated otherwise.

The term “alkynyl”, refers to an organic group that is comprised of carbon and hydrogen atoms that contains a triple covalent bond between two carbons. Typically, an “alkynyl” as used in this disclosure, refers to organic group that contains that contains 2, 3, 4, 5, 6, 7, 8, 9, 10, 11, 12, 13, 14, 15, 16, 17, 18, 19, 20, or 30 carbon atoms, or any range of carbon atoms between or including any two of the foregoing values. While a C2-alkynyl can form a triple bond to a carbon of a parent chain, an alkynyl group of three or more carbons can contain more than one triple bond.

Where if there is more than 3 carbon, the carbons may be connected in a linear manner, or alternatively if there are more than 4 carbons then the carbons may also be linked in a branched fashion so that the parent chain contains one or more secondary, tertiary, or quaternary carbons. An alkynyl may be substituted or unsubstituted, unless stated otherwise.

The term “aryl”, as used in this disclosure, refers to a conjugated planar ring system with delocalized pi electron clouds that contain only carbon as ring atoms. An “aryl” for the purposes of this disclosure encompass from 1 to 4 aryl rings wherein when the aryl is greater than 1 ring the aryl rings are joined so that they are linked, fused, or a combination thereof. An aryl may be substituted or unsubstituted, or in the case of more than one aryl ring, one or more rings may be unsubstituted, one or more rings may be substituted, or a combination thereof.

The term “cycloalkyl”, as used in this disclosure, refers to an alkyl that contains at least 3 carbon atoms but no more than 12 carbon atoms connected so that it forms a ring. A “cycloalkyl” for the purposes of this disclosure encompasses from 1 to 4 cycloalkyl rings, wherein when the cycloalkyl is greater than 1 ring, then the cycloalkyl rings are joined so that they are linked, fused, or a combination thereof. A cycloalkyl may be substituted or unsubstituted, or in the case of more than one cycloalkyl ring, one or more rings may be unsubstituted, one or more rings may be substituted, or a combination thereof.

The term “hetero-” when used as a prefix, such as, hetero-alkyl, hetero-alkenyl, hetero-alkynyl, or hetero-hydrocarbon, for the purpose of this disclosure refers to the specified hydrocarbon having one or more carbon atoms replaced by non-carbon atoms as part of the parent chain. Examples of such non-carbon atoms include, but are not limited to, N, O, S, Si, Al, B, and P. If there is more than one non-carbon atom in the hetero-based parent chain then this atom may be the same element or may be a combination of different elements, such as N and O. In a particular embodiment, a “hetero”-hydrocarbon (e.g., alkyl, alkenyl, alkynyl) refers to a hydrocarbon that has from 1 to 3 C, N and/or S atoms as part of the parent chain.

The term “heterocycle,” as used herein, refers to ring structures that contain at least 1 noncarbon ring atom. A “heterocycle” for the purposes of this disclosure encompass from 1 to 4 heterocycle rings, wherein when the heterocycle is greater than 1 ring the heterocycle rings are joined so that they are linked, fused, or a combination thereof. A heterocycle may be aromatic or nonaromatic, or in the case of more than one heterocycle ring, one or more rings may be nonaromatic, one or more rings may be aromatic, or a combination thereof. A heterocycle may be substituted or unsubstituted, or in the case of more than one heterocycle ring one or more rings may be unsubstituted, one or more rings may be substituted, or a combination thereof. Typically, the noncarbon ring atom is N, O, S, Si, Al, B, or P. In the case where there is more than one noncarbon ring atom, these noncarbon ring atoms can either be the same element, or combination of different elements, such as N and O.

Examples of heterocycles include, but are not limited to: a monocyclic heterocycle such as, aziridine, oxirane, thiirane, azetidine, oxetane, thietane, pyrrolidine, pyrroline, imidazolidine, pyrazolidine, pyrazoline, dioxolane, sulfolane 2,3-dihydrofuran, 2,5-dihydrofuran tetrahydrofuran, thiophane, piperidine, 1,2,3,6-tetrahydro-pyridine, piperazine, morpholine, thiomorpholine, pyran, thiopyran, 2,3-dihydropyran, tetrahydropyran, 1,4-dihydropyridine, 1,4-dioxane, 1,3-dioxane, dioxane, homopiperidine, 2,3,4,7-tetrahydro-1H-azepine, homopiperazine, 1,3-dioxepane, 4,7-dihydro-1,3-dioxepin, and hexamethylene oxide; and polycyclic heterocycles such as, indole, indoline, isoindoline, quinoline, tetrahydroquinoline, isoquinoline, tetrahydroisoquinoline, 1,4-benzodioxan, coumarin, dihydrocoumarin, benzofuran, 2,3-dihydrobenzofuran, isobenzofuran, chromene, chroman, isochroman, xanthene, phenoxathiin, thianthrene, indolizine, isoindole, indazole, purine, phthalazine, naphthyridine, quinoxaline, quinazoline, cinnoline, pteridine, phenanthridine, perimidine, phenanthroline, phenazine, phenothiazine, phenoxazine, 1,2-benzisoxazole, benzothiophene, benzoxazole, benzthiazole, benzimidazole, benztriazole, thioxanthine, carbazole, carboline, acridine, pyrolizidine, and quinolizidine. In addition to the polycyclic heterocycles described above, heterocycle includes polycyclic heterocycles wherein the ring fusion between two or more rings includes more than one bond common to both rings and more than two atoms common to both rings. Examples of such bridged heterocycles include quinuclidine, diazabicyclo[2.2.1]heptane and 7-oxabicyclo[2.2.1]heptane.

The terms “heterocyclic group”, “heterocyclic moiety”, “heterocyclic”, or “heterocyclo” used alone or as a suffix or prefix, refers to a heterocycle that has had one or more hydrogens removed there from.

The term “hydrocarbons” refers to groups of atoms that contain only carbon and hydrogen. Examples of hydrocarbons that can be used in this disclosure include, but are not limited to, alkanes, alkenes, alkynes, arenes, and benzyls.

The term “optionally substituted” means independent replacement of one or more hydrogen atoms with a substituent. The term “optionally substituted” also refers to a functional group, typically a hydrocarbon or heterocycle, where one or more hydrogen atoms may be replaced with a substituent. Accordingly, “optionally substituted” refers to a functional group that is substituted, in that one or more hydrogen atoms are replaced with a substituent, or unsubstituted, in that the hydrogen atoms are not replaced with a substituent. For example, an optionally substituted hydrocarbon group refers to an unsubstituted hydrocarbon group or a substituted hydrocarbon group.

As described herein, the subject is administered a composition comprising one or more human milk oligosaccharides. The composition can comprise 10% or more, 20% or more, 30% or more, 40% or more, or 50% or more by mass one or more human milk oligosaccharide. In certain aspects, the composition is not human milk. In additional aspects, the composition is not derived from human milk.

In certain aspects, the one or more human milk oligosaccharides are selected from lacto-N-tetraose (LNT), lacto-N-neotetraose (LNnT), lacto-N-hexaose (LNH), lacto-N-neohexaose (LNnH), 2′fucosyllactose (2′FL), 3′fucosyllacose (3′FL), lacto-difucotetraose (LDFT), lacto-N-fucopenaose (LNFP II/III), lactose-N-fucopentaose I (LNFP I), lacto-N-difuco-hexaose I (LNDFH I), lacto-N-difuco-hexaose II (LNDFH II), difucosyl-para-lacto-N-neohexaose (DFpLNnH), difucosyllacto-N-hexaose c (DFLNH c), 3′ sialyllactose (3′SL), 6′ sialyllactose (6′SL), LS-tetrasaccharide a (LSTa), LS-tetrasaccharide b (LST b), LS-tetrasaccharide c (LST c), 3′-sialyl-N-acetyllactosamine (3′ SLN), 6′-sialyl-N-acetyllactosamine (6′ SLN), or disialyllacto-N-tetraose (DSLNT), or a combination of any of thereof. In yet other aspects, the one or more human milk oligosaccharides are selected from 2′FL, 3′FL, 3′SL, 6′SL, LNT, or LNnT, or a combination of any of thereof.

The composition administered to the subject can comprise one HMO or can comprise a mixture of two, three, four, five or more HMOs. In certain aspects, the composition comprises one HMO and the HMO is selected from the group consisting of 2′FL, 3′FL, 3′SL, 6′SL, LNT, or LNnT.

In further aspects, the composition comprises a mixture of 2′FL and at least one other HMO. In certain aspects, the composition comprises 2′FL and LNT; 2′FL and LNnT; 2′FL, 3′FL, 3′SL, 6′SL and LNT. The composition comprising 2′FL and LNT includes a 4:1 mixture of 2′FL and LNT; such a composition is GRAS (generally regarded as safe) and is available from Glycom, Lyngby, Denmark. A composition comprising 2′FL, 3′FL, 3′SL, 6′SL and LNT is sold by Jennewein Biotechnologie and is GRAS.

In further aspects, the composition comprises a mixture of one neutral core and one neutral fucosylated human milk oligosaccharide. In additional embodiments, the composition comprises a mixture of one neutral and one acidic human milk oligosaccharide.

The composition can be administered orally. The composition can be formulated as a liquid formulation (e.g., aqueous solutions), a powder, a nutritional additive, protein bar, as well as a tablet and capsule. In certain aspects, the composition is an aqueous solution wherein the water in the solution is water recycled from urine using urine processors or urine recycling systems in the spacecraft (e.g., the space shuttle). HMOs are excreted in the urine and thus, the HMOs used in the present method can be recycled from urine.

Human milk oligosaccharides, including 2′-fucosyllactose, 3′ sialyllactose, and 6′ sialyllactose, can be readily prepared with well-established synthetic biology methods.

In some embodiments, the composition comprises at least 9% (e.g., 10, 15, 20, 25, 30, 35, 40, 45, 50, 55, 60, 65, 70, 75, 80, 85, 90, 95, or 100%; or any value between any of the foregoing) of the total human milk oligosaccharides in the composition.

In certain aspects, the pharmaceutical composition one or more human milk oligosaccharides, and optionally a pharmaceutically acceptable carrier or excipient.

The oligosaccharide can be administered in an amount from about 1 g to about 20 g, about 1 to about 15 g, or about 2.5 to 7.5 g per day or per dose. The oligosaccharide can be administered at various intervals, for example, once a day, twice a day, three times a day, once a week, twice a week, or as needed.

In further aspects, the methods additionally provide an improvement in the subject's microbiota (gut and/or oral) composition. Improvement in, or avoidance of, gastrointestinal symptoms, such as constipation, diarrhea, stool consistency, stool smell, flatulence and abdominal pain is desirable, such as, for example, at weeks 8 and 16 of the beginning of treatment.

Oral administration of the oligosaccharides of the disclosure provide for systemic circulation of the oligosaccharides both in infants and adults. Unlike other drug products approved by the FDA, the oligosaccharides described herein can not only be administered to treat a disease or disorder in an adult subject, but can also be administered to pregnant females, infants, and subjects who have impaired organ function (e.g., liver disfunction, kidney failure). Due to the oligosaccharides of the disclosure having little to no adverse effects in humans, this form of therapy could be used as a preventive, as a first line therapy option, or as an adjunct to existing therapies that would be well tolerated by patients of either sex.

In a further embodiment, said oligosaccharide is substantially a single enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, substantially an individual diastereomer, or a mixture of about 90% or more by weight of an individual diastereomer and about 10% or less by weight of any other diastereomer.

The oligosaccharides disclosed herein may be enantiomerically pure, such as a single enantiomer or a single diastereomer, or be stereoisomeric mixtures, such as a mixture of enantiomers, a racemic mixture, or a diastereomeric mixture. As such, one of skill in the art will recognize that administration of an oligosaccharide in its (R) form is equivalent, for oligosaccharides that undergo epimerization in vivo, to administration of the oligosaccharide in its (S) form. Conventional techniques for the preparation/isolation of individual enantiomers include chiral synthesis from a suitable optically pure precursor or resolution of the racemate using, for example, chiral chromatography, recrystallization, resolution, diastereomeric salt formation, or derivatization into diastereomeric adducts followed by separation.

When the oligosaccharide disclosed herein contains an acidic or basic moiety, it may also be disclosed as a pharmaceutically acceptable salt (See, Berge et al., J. Pharm. Sci. 1977, 66, 1-19; and “Handbook of Pharmaceutical Salts, Properties, and Use,” Stah and Wermuth, Ed.; Wiley-VCH and VHCA, Zurich, 2002).

Suitable acids for use in the preparation of pharmaceutically acceptable salts include, but are not limited to, acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxo-glutaric acid, glycolic acid, hippuric acid, hydrobromic acid, hydrochloric acid, hydroiodic acid, (+)-L-lactic acid, (±)-DL-lactic acid, lactobionic acid, lauric acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid.

Suitable bases for use in the preparation of pharmaceutically acceptable salts, including, but not limited to, inorganic bases, such as magnesium hydroxide, calcium hydroxide, potassium hydroxide, zinc hydroxide, or sodium hydroxide; and organic bases, such as primary, secondary, tertiary, and quaternary, aliphatic and aromatic amines, including L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, secondary amines, triethanolamine, trimethylamine, triethylamine, N-methyl-D-glucamine, 2-amino-2-(hydroxymethyl)-1,3-propanediol, and tromethamine.

The oligosaccharide as disclosed herein may also be designed as a prodrug, which is a functional derivative of the oligosaccharide as disclosed herein and is readily convertible into the parent oligosaccharide in vivo. Prodrugs are often useful because, in some situations, they may be easier to administer than the parent oligosaccharide. They may, for instance, be bioavailable by oral administration whereas the parent oligosaccharide is not.

The prodrug may also have enhanced solubility in pharmaceutical compositions over the parent oligosaccharide. A prodrug may be converted into the parent drug by various mechanisms, including enzymatic processes and metabolic hydrolysis. See Harper, Progress in Drug Research 1962, 4, 221-294; Morozowich et al. in “Design of Biopharmaceutical Properties through Prodrugs and Analogs,” Roche Ed., APHA Acad. Pharm. Sci. 1977; “Bioreversible Carriers in Drug in Drug Design, Theory and Application,” Roche Ed., APHA Acad. Pharm. Sci. 1987; “Design of Prodrugs,” Bundgaard, Elsevier, 1985; Wang et al., Curr. Pharm. Design 1999, 5, 265-287; Pauletti et al., Adv. Drug. Delivery Rev. 1997, 27, 235-256; Mizen et al., Pharm. Biotech. 1998, 11, 345-365; Gaignault et al., Pract. Med. Chem. 1996, 671-696; Asgharnejad in “Transport Processes in Pharmaceutical Systems,” Amidon et al., Ed., Marcell Dekker, 185-218, 2000; Balant et al., Eur. J. Drug Metab. Pharmacokinet. 1990, 15, 143-53; Balimane and Sinko, Adv. Drug Delivery Rev. 1999, 39, 183-209; Browne, Clin. Neuropharmacol. 1997, 20, 1-12; Bundgaard, Arch. Pharm. Chem. 1979, 86, 1-39; Bundgaard, Controlled Drug Delivery 1987, 17, 179-96; Bundgaard, Adv. Drug Delivery Rev. 1992, 8, 1-38; Fleisher et al., Adv. Drug Delivery Rev. 1996, 19, 115-130; Fleisher et al., Methods Enzymol. 1985, 112, 360-381; Farquhar et al., J. Pharm. Sci. 1983, 72,324-325; Freeman et al., J. Chem. Soc., Chem. Commun. 1991, 875-877; Friis and Bundgaard, Eur. J. Pharm. Sci. 1996, 4, 49-59; Gangwar et al., Des. Biopharm. Prop. Prodrugs Analogs, 1977, 409-421; Nathwani and Wood, Drugs 1993, 45, 866-94; Sinhababu and Thakker, Adv. Drug Delivery Rev. 1996, 19, 241-273; Stella et al., Drugs 1985, 29, 455-73; Tan et al., Adv. Drug Delivery Rev. 1999, 39, 117-151; Taylor, Adv. Drug Delivery Rev. 1996, 19, 131-148; Valentino and Borchardt, Drug Discovery Today 1997, 2, 148-155; Wiebe and Knaus, Adv. Drug Delivery Rev. 1999, 39, 63-80; Waller et al., Br. J. Clin. Pharmac. 1989, 28, 497-507.

The oligosaccharide may be produced by biotechnological means using enzyme-based fermentation technology (recombinant or natural enzymes) or microbial fermentation technology. In the latter case, microbes may either express their natural enzymes and substrates or may be engineered to produce respective substrates and enzymes. Single microbial cultures and/or mixed cultures may be used. Alternatively, the oligosaccharides may be produced by chemical synthesis from lactose and other substrates.

Biotechnological approaches have made it possible for the large scale, cost-efficient production of target oligosaccharides.

Precisely, the oligosaccharides disclosed herein can be produced in high yields in aqueous media by fermentation of genetically modified bacteria, yeasts or other microorganisms. See, for example, WO200104341; WO2007101862, WO2010070104; WO2010142305; WO2012112777; Priem et al., Glycobiology 12:235 (2002); Drouillard et al., Angew. Chem. Int. Ed. 45:1778 (2006); Han et al., Biotechnol. Adv. 30:1268 (2012); Lee et al., Microb. Cell Fact. 11:48 (2012); Baumgartner et al., Microb. Cell Fact. 12:40 (2013); and WO 2014135167A1.

Alternatively, the oligosaccharides of the disclosure can be synthesized based upon methods described in WO2011100980A1; WO2012007588A1; WO2012127410A1; WO2012155916A1; WO2013044928A1; and U.S. Pat. No. 9,102,966B2. 2′-FL can be made as described in WO 2010/115934 and WO 2010/115935, 3-FL can be made as described in WO 2013/139344. Fucosylated oligosaccharides can be made as described in WO 2012/127410. With regard to biotechnological methods, WO 2001/04341 and WO 2007/101862 describe how to make oligosaccharides optionally substituted by fucose using genetically modified E. coli. The oligosaccharides disclosed herein can be produced in high yields in aqueous media by fermentation of genetically modified bacteria, yeasts or other microorganisms. See, for example, WO200104341; WO2007101862, WO2010070104; WO2010142305; WO2012112777; Priem et al., Glycobiology 12:235 (2002); Drouillard et al., Angew. Chem. Int. Ed. 45:1778 (2006); Han et al., Biotechnol. Adv. 30:1268 (2012); Lee et. al., Microb. Cell Fact. 11:48 (2012); Baumgartner et al., Microb. Cell Fact. 12:40 (2013); and WO2014135167A1.

Alternatively, the oligosaccharides of the disclosure can be synthesized based upon methods described in WO2011100980A1; WO2012007588A1;WO2012127410A1; WO2012155916A1;WO2013044928A1; and U.S. Pat. No. 9,102,966B2. 2′-FL can be made as described in WO 2010/115934 and WO 2010/115935, 3-FL can be made as described in WO 2013/139344, 6′-SL and salts thereof can be made as described in WO 2010/100979, sialylated oligosaccharides can be made as described in WO 2012/113404 and mixtures of human milk oligosaccharides can be made as described in WO 2012/113405. As examples of enzymatic production, sialylated oligosaccharides can be made as described in WO 2012/007588, fucosylated oligosaccharides can be made as described in WO 2012/127410.

With regards to biotechnological methods, WO 2001/04341 and WO 2007/101862 describe how to make oligosaccharides optionally substituted by fucose or sialic acid using genetically modified E. coli.

In a certain embodiment, the composition descried herein can further comprise one or more foodgrade agents. Examples of foodgrade agents that can be used with the oligosaccharides disclosed herein, include, but are not limited to, milk (e.g., cow's milk, almond milk, soy milk), yogurt, maltodextrin, milk protein concentrate, Sucromalt, glycerine, cocoa powder, soy protein isolate, fructose, vegetable or animal oils (e.g., high oleic safflower oil, soy oil, canola oil), plant sterol esters, HMSs/HMOs, soy lecithin, carrageenan, taurine, L-carnitine, vitamins and/or minerals (e.g., sodium ascorbate, potassium citrate, sodium phosphate, calcium citrate, choline chloride, potassium chloride, sodium citrate, magnesium oxide, alpha-tocopheryl acetate, zinc sulfate, ferrous sulfate, niacinamide, calcium pantothenate, vitamin A palmitate, citric acid, manganese sulfate, pyridoxine hydrochloride, vitamin D3, copper sulfate, thiamine mononitrate, riboflavin, beta carotene, folic acid, biotin, potassium iodide, chromium chloride, sodium selenate, sodium molybdate, phytonadione, vitamin B12, magnesium chloride, calciumphosphate).

Disclosed herein are pharmaceutical compositions comprising one or more oligosaccharides of the disclosure, or a pharmaceutically acceptable salt, solvate, or prodrug thereof, as an active ingredient, combined with a pharmaceutically acceptable vehicle, carrier, diluent, or excipient, or a mixture thereof; in combination with one or more pharmaceutically acceptable excipients or carriers.

Disclosed herein are pharmaceutical compositions in modified release dosage forms, which comprise one or more oligosaccharides of the disclosure, or a pharmaceutically acceptable salt, solvate, or prodrug thereof; and one or more release controlling excipients or carriers as described herein. Suitable modifiedrelease dosage vehicles include, but are not limited to, hydrophilic or hydrophobic matrix devices, water-soluble separating layer coatings, enteric coatings, osmotic devices, multiparticulate devices, and combinations thereof.

The pharmaceutical compositionsmay also comprise non-release controlling excipients or carriers.

Further disclosed herein are pharmaceutical compositions in enteric coated dosage forms, which comprise one or more human milk oligosaccharides as disclosed herein, or a pharmaceutically acceptable salt, solvate, or prodrug thereof; and one or more release controlling excipients or carriers for use in an enteric coated dosage form. The pharmaceutical compositions may also comprise non-release controlling excipients or carriers.

Further disclosed herein are pharmaceutical compositions in effervescent dosage forms, which comprise one or more human milk oligosaccharides as disclosed herein in substantially pure form (e.g., lacking other oligosaccharides found in milk), or a pharmaceutically acceptable salt, solvate, or prodrug thereof; and one or more release controlling excipients or carriers for use in an effervescent dosage form. The pharmaceutical compositions may also comprise non-release controlling excipients or carriers.

Additionally, disclosed are pharmaceutical compositions in a dosage form that has an instant releasing component and at least one delayed releasing component, and is capable of giving a discontinuous release of one or more human milk oligosaccharides disclosed herein in the form of at least two consecutive pulses separated in time (e.g., separated in time from 0.1 up to 24 hours or a few days). The pharmaceutical compositions comprise an oligosaccharide as disclosed herein, or a pharmaceutically acceptable salt, solvate, or prodrug thereof; and one or more release controlling and non-release controlling excipients or carriers, such as those excipients or carriers suitable for a disruptable semi-permeable membrane and as swellable substances.

Disclosed herein also are pharmaceutical compositions in a dosage form for oral administration to a subject, which comprise one or more as disclosed herein, or a pharmaceutically acceptable salt, solvate, or prodrug thereof; and one or more pharmaceutically acceptable excipients or carriers, enclosed in an intermediate reactive layer comprising a gastric juice-resistant polymeric layered material partially neutralized with alkali and having cation exchange capacity and a gastric juice-resistant outer layer.

Provided herein are pharmaceutical compositions that comprise about 0.1 to about 1000 mg or up to 2000 mg or up to 3000 mg (or any value between 0.1-3000 mg), about 1 to about 500 mg, about 2 to about 100 mg, about 1 mg, about 2 mg, about 3 mg, about 5 mg, about 10 mg, about 15 mg, about 20 mg, about 30 mg, about 40 mg, about 50 mg, about 100 mg, about 500 mg of one or more oligosaccharides as disclosed herein, in the form of immediate release tablets for oral administration. The pharmaceutical compositions further comprise inactive ingredients such as flavoring agents, copovidone, ethylcellulose, magnesium stearate, mannitol, and silicondioxide.

Provided herein are pharmaceutical compositions that comprise about 0.1 to about 1000 mg or up to 2000 mg or up to 3000 mg (or any value there between), about 1 to about 500 mg, about 2 to about 100 mg, about 1 mg, about 2 mg, about 3 mg, about 5 mg, about 10 mg, about 15 mg, about 20 mg, about 30 mg, about 40 mg, about 50 mg, about 100 mg, about 500 mg of one or more oligosaccharides as disclosed herein, in the form of extended release tablets for oral administration. The pharmaceutical compositions further comprise inactive ingredients such as ethylcellulose, dibutyl sebacate, polyvinyl pyrroliodone, sodium stearyl fumarate, colloidal silicon dioxide, and polyvinyl alcohol.

The pharmaceutical compositions disclosed herein may be disclosed in unit-dosage forms or multiple-dosage forms. Unit-dosage forms, as used herein, refer to physically discrete units suitable for administration to human and animal subjects and packaged individually as is known in the art. Each unit-dose contains a predetermined quantity of the oligosaccharide sufficient to produce the desired therapeutic effect, in association with the required pharmaceutical carriers or excipients. Examples of unit-dosage forms include ampoules, syringes, and individually packaged to capsules. Unit-dosage forms may be administered in fractions or multiples thereof. A multiple-dosage form is a plurality of identical unit-dosage forms packaged in a single container to be administered in segregated unit-dosage form. Examples of multiple-dosage forms include vials, bottles of tablets or capsules, or bottles of pints or gallons.

The oligosaccharides as disclosed herein may be administered alone, or in combination with one or more other oligosaccharides disclosed herein, and/or one or more other active ingredients. The pharmaceutical compositions that comprise an oligosaccharide disclosed herein may be formulated in various dosage forms for oral, parenteral, and topical administration. The pharmaceutical compositions may also be formulated as a modified release dosage form, including delayed-, extended-, prolonged-, sustained-, pulsatile-, controlled-, accelerated- and fast-, targeted-, programmed-release, and gastric retention dosage forms.

These dosage forms can be prepared according to conventional methods and techniques known to those skilled in the art (see, Remington: The Science and Practice of Pharmacy, supra; Modified-Release Drug Delivery Technology, Rathbone et al., Eds., Drugs and the Pharmaceutical Science, Marcel Dekker, Inc.: New York, N.Y., 2002; Vol. 126).

The pharmaceutical compositions disclosed herein may be administered at once, or multiple times at intervals of time. It is understood that the precise dosage and duration of treatment may vary with the age, weight, and condition of the patient being treated, and may be determined empirically using known testing protocols or by extrapolation from in vivo or in vitro test or diagnostic data. It is further understood that for any particular individual, specific dosage regimens should be adjusted over time according to the individual need and the professional judgment of the person administering or supervising the administration of the formulations.

Once improvement of the patient's condition has occurred, a maintenance dose is administered if necessary. Subsequently, the dosage or the frequency of administration, or both, can be reduced, as a function of the symptoms, to a level at which the improved disease, disorder or condition is retained. Patients can, however, require intermittent treatment on a long-term basis upon any recurrence of symptoms.

The pharmaceutical compositions disclosed herein may be formulated in solid, semisolid, or liquid dosage forms for oral administration. As used herein, oral administration also includes buccal, lingual, and sublingual administration. Suitable oral dosage forms include, but are not limited to, tablets, capsules, pills, troches, lozenges, pastimes, cachets, pellets, medicated chewing gum, granules, bulk powders, effervescent or non-effervescent powders or granules, solutions, emulsions, suspensions, solutions, wafers, sprinkles, elixirs, and syrups. In addition to the oligosaccharides, the pharmaceutical compositions may contain one or more pharmaceutically acceptable carriers or excipients, including, but not limited to, binders, fillers, diluents, disintegrants, wetting agents, lubricants, glidants, coloring agents, dye-migration inhibitors, sweetening agents, and flavoring agents.

Binders or granulators impart cohesiveness to a tablet to ensure the tablet remaining intact after compression. Suitable binders or granulators include, but are not limited to, starches, such as corn starch, potato starch, and pre-gelatinized starch (e.g., STARCH 1500); gelatin; sugars, such as sucrose, glucose, dextrose, molasses, and lactose; natural and synthetic gums, such as acacia, alginic acid, alginates, extract of Irish moss, Panwar gum, ghatti gum, mucilage of isabgol husks, carboxymethyl cellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxyethyl cellulose (HEC), hydroxypropyl cellulose (HPC), hydroxypropyl methyl cellulose (HPMC); microcrystalline celluloses, such as AVICEL-PH-101, AVICEL-PH-103, AVICEL RC-581, AVICEL-PH-105 (FMC Corp., Marcus Hook, Pa.); and mixtures thereof. Suitable fillers include, but are not limited to, talc, calcium carbonate, microcrystalline cellulose, powdered cellulose, dextrates, kaolin, mannitol, silicic acid, sorbitol, starch, pre-gelatinized starch, and mixtures thereof. The binder or filler may be present from about 50 to about 99% by weight in the pharmaceutical compositions disclosed herein.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such asmannitol, lactose, sorbitol, sucrose, and inositol, when present in sufficient quantity, can impart properties to some compressed tablets that permit disintegration in the mouth by chewing. Such compressed tablets can be used as chewable tablets.

Suitable disintegrants include, but are not limited to, agar; bentonite; celluloses, such as methylcellulose and carboxymethylcellulose; wood products; natural sponge; cation-exchange resins; alginic acid; gums, such as guar gum and Veegum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; aligns; and mixtures thereof. The amount of disintegrant in the pharmaceutical compositions disclosed herein varies upon the type of formulation, and is readily discernible to those of ordinary skill in the art. The pharmaceutical compositions disclosed herein may contain from about 0.5 to about 15% or from about 1 to about 5% by weight of a disintegrant.

It should be understood that many carriers and excipients may serve several functions, even within the same formulation. The pharmaceutical compositions disclosed herein may be formulated as compressed tablets, tablet triturates, chewable lozenges, rapidly dissolving tablets, multiple compressed tablets, or enteric-coating tablets, sugar-coated, or film-coated tablets.

The tablet dosage forms may be prepared from the active ingredient in powdered, crystalline, or granular forms, alone or in combination with one or more carriers or excipients described herein, including binders, disintegrants, controlled-release polymers, lubricants, diluents, and/or colorants. Flavoring and sweetening agents are especially useful in the formation of chewable tablets and lozenges.

The pharmaceutical compositions disclosed herein may be formulated as soft or hard capsules, which can be made from gelatin, methylcellulose, starch, or calcium alginate. The hard gelatin capsule, also known as the dry-filled capsule (DFC), consists of two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms disclosed herein may be encapsulated in a capsule. Suitable liquid and semisolid dosage forms include solutions and suspensions in propylene carbonate, vegetable oils, or triglycerides. Capsules containing such solutions can be prepared as described in U.S. Pat. Nos. 4,328,245; 4,409,239; and 4,410,545. The capsules may also be coated as known by those of skill in the art in order to modify or sustain dissolution of the activeingredient.

The pharmaceutical compositions disclosed herein may be formulated in liquid and semisolid dosage forms, including emulsions, solutions, suspensions, elixirs, and syrups. An emulsion is a two-phase system, in which one liquid is dispersed in the form of small globules throughout another liquid, which can be oil-in-water or water-in-oil. Emulsions may includea pharmaceutically acceptable non-aqueous liquids or solvent, emulsifying agent, and preservative.

Suspensions may include a pharmaceutically acceptable suspending agent and preservative. Aqueous alcoholic solutions may include a pharmaceutically acceptable acetal, such as a di(lower alkyl) acetal of a lower alkyl aldehyde (the term “lower” means an alkyl having between 1 and 6 carbon atoms), e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

Other useful liquid and semisolid dosage forms include, but are not limited to, those containing the active ingredient(s) disclosed herein, and a dialkylated mono- or poly-alkylene glycol.

The pharmaceutical compositions disclosed herein for oral administration may be also formulated in the forms of liposomes, micelles, microspheres, or nanosystems. Micellar dosage forms can be prepared as described in U.S. Pat. No. 6,350,458.

The pharmaceutical compositions disclosed herein may be formulated as non-effervescent or effervescent, granules and powders, to be reconstituted into a liquid dosage form. Pharmaceutically acceptable carriers and excipients used in the non-effervescent granules or powders may include diluents, sweeteners, and wetting agents. Pharmaceutically acceptable carriers and excipients used in the effervescent granules or powders may include organic acids and a source of carbon dioxide.

The pharmaceutical compositions disclosed herein can be formulated as an oral nutritional composition. An oral nutritional composition can contain sources of protein, lipids and/or digestible carbohydrates and can be in solid, powdered or liquid forms. The composition can be designed to be the sole source of nutrition or a nutritional supplement. Suitable protein sourcesinclude intact, hydrolyzed, and partially hydrolyzed protein, which can be derived from any suitable source such as milk (e.g., casin, whey), animal (e.g., meat, fish), cereal (e.g., rice, corn), and vegetable (e.g., soy, potato, pea), insect (e.g., locust) and combinations of these sources.

Examples of the source of protein include whey protein concentrates, whey protein isolate, whey protein hydrolysates, and acid.

The pharmaceutical compositions disclosed herein may be formulated as immediate or modified release dosage forms, including delayed-, sustained, pulsed-, controlled, targeted-, and programmed-release forms.

The pharmaceutical compositions disclosed herein may be co-formulated with other active ingredients which do not impair the desired therapeutic action, or with substances that supplement the desired action.

The pharmaceutical compositions disclosed herein may be administered parenterally by injection, infusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, and subcutaneous administration.

The pharmaceutical compositions disclosed herein may be formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science (see, Remington: The Science and Practice of Pharmacy, supra).

The pharmaceutical compositions intended for parenteral administration may include one or more pharmaceutically acceptable carriers and excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

The pharmaceutical compositions disclosed herein may be formulated for single or multiple dosage administration. The single dosage formulations are packaged in an ampule, a vial, or a syringe. The multiple dosage parenteral formulations must contain an antimicrobial agent at bacteriostatic or fungistatic concentrations. All parenteral formulations must be sterile, as known and practiced in the art.

The pharmaceutical compositions may be formulated as a suspension, solid, semi-solid, or thixotropic liquid, for administration as an implanted depot. In one embodiment, the pharmaceutical compositions disclosed herein are dispersed in a solid inner matrix, which is surrounded by an outer polymeric membrane that is insoluble in body fluids but allows the active ingredient in the pharmaceutical compositions diffuse through.

Pharmaceutically acceptable carriersand excipients suitable for use in the topical formulations disclosed herein include, but are not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, penetration enhancers, cryoprotectants, lyoprotectants, thickening agents, and inert gases.

The pharmaceutical compositions disclosed herein may be administered intranasally or by inhalation to the respiratory tract. The pharmaceutical compositions may be formulated in the form of an aerosol or solution for delivery using a pressurized container, pump, spray, atomizer, such as an atomizer using electrohydrodynamics to produce a fine mist, or nebulizer, alone or in combination with a suitable propellant, such as 1,1,1,2-tetrafluoroethane or 1,1,1,2,3,3,3-heptafluoropropane. The pharmaceutical compositions may also be formulated as a dry powder for insufflation, alone or in combination with an inert carrier such as lactose or phospholipids; and nasal drops. For intranasal use, the powder may comprise a bioadhesive agent, including chitosan or cyclodextrin.

The pharmaceutical compositions disclosed herein may be formulated as a modified release dosage form. As used herein, the term “modified release” refers to a dosage form in which the rate or place of release of the active ingredient(s) is different from that of an immediate dosage form when administered by the same route.

Modified release dosage forms include delayed-, extended-, prolonged-, sustained-, pulsatile-, controlled-, accelerated- and fast-, targeted-, programmed-release, and gastric retention dosage forms. The pharmaceutical compositions in modified release dosage forms can be prepared using a variety of modified release devices and methods known to those skilled in the art, including, but not limited to, matrix controlled release devices, osmotic controlled release devices, multiparticulate controlled release devices, ion-exchange resins, enteric coatings, multilayered coatings, microspheres, liposomes, and combinations thereof. The release rate of the active ingredient(s) can also be modified by varying the particle sizes and polymorphism of the active ingredient(s).

The pharmaceutical compositions disclosed herein in a modified release dosage form may be prepared by methods known to those skilled in the art, including direct compression, dry or wet granulation followed by compression, melt-granulation followed by compression.

Generally, the amount of an oligosaccharide disclosed herein required to be administered to the person can vary depending upon factors such as the risk and condition severity, the age of the person, the form of the composition, and other medications being administered to the person. It would be expected that an oligosaccharide described herein should be well tolerated irrespective of the age and condition of the subject. The dosage of oligosaccharide to be administered can readily be set by a medical practitioner and would generally be in the range from about 10 mg to about 20 g per day, in certain embodiments from about 10 mg to about 15 g per day, from about 100 mg to about 10 g per day, in certain embodiments from about 500 mg to about 10 g per day, in certain embodiments from about 1 g to about 7.5 g per day. An appropriate dose can be determined based on several factors, including, for example, the body weight and/or condition of the patient being treated, the severity of the condition, being treated, other ailments and/or diseases of the person, the incidence and/or severity of side effects and the manner of administration. Appropriate dose ranges can be determined by methods known to those skilled in the art. During an initial treatment phase,the dosing can be higher (for example 200 mg to 20 g per day, preferably 500 mg to 15 g per day, more preferably 1 g to 10 g per day, in certain embodiments 2.5 g to 7.5 g per day). During a maintenance phase, the dosing can be reduced (for example, 10 mg to 10 g per day, preferably 100 mg to 7.5 g per day, more preferably 500 mg to 5 g per day, in certain embodiments 1 g to 2.5 g per day).

Depending on the disorder to be treated and the injection in suitable dosage unit with pharmaceutically acceptable carriers, adjuvants and vehicles appropriate for each route of administration.

The dose may be in the form of one, two, three, four, five, six, or more sub-doses that are administered at appropriate intervals per day. The dose or sub-doses can be administered in the form of dosage units containing from about 0.01 to about 2 grams, from about 0.05 to about 1 gram, or from about 10 to about 500 milligrams active ingredient(s) per dosage unit.

In certain embodiments, an appropriate dosage level is about 0.01 to about 5 g/kg patient body weight per day (mg/kg per day), about 0.01 to about 1 g/kg per day, about 0.01 to about 0.5 g/kg per day, or about 0.1 to about 500 mg/kg per day, which may be administered in single ormultiple doses. A suitable dosage level may be about 0.1 to about 500 mg/kg per day, about 0.1 to about 250 mg/kg per day, or about 0.1 to about 100 mg/kg per day. Within this range the dosage may be about 0.01 to about 0.1, about 0.1 to about 1.0, about 1.0 to about 10, or about 10 to about 100 mg/kg per day.

The oligosaccharides disclosed herein may also be combined or used in combination with other agents useful in the treatment, prevention, or amelioration of one or more symptoms of a condition as described herein. Or, by way of example only, the therapeutic effectiveness of one of the oligosaccharides described herein may be enhanced by administration of an adjuvant (i.e., by itself the adjuvant may only have minimal therapeutic benefit, but in combination with another therapeutic agent, the overall therapeutic benefit to the patient is enhanced).

Such other agents, adjuvants, or drugs, may be administered, by a route and in an amount commonly used therefore, simultaneously or sequentially with an oligosaccharide as disclosed herein. When an oligosaccharide as disclosed herein is used contemporaneously with one or more other drugs, a pharmaceutical composition containing such other drugs in addition to an oligosaccharide disclosed herein may be utilized but is not required.

Accordingly, the pharmaceutical compositions disclosed herein include those that also contain one or more other active ingredients or therapeutic agents, in addition to an oligosaccharide disclosed herein.

### EXAMPLES

The compositions and methods of the invention can be evaluated in one or more models described in Zwart et al. (2010), Capacity of Omega-3 Fatty Acids or Eicosapentaenoic Acid to Counteract Weightlessness-Induced Bone Loss by Inhibiting NF-κB Activation: From Cells to Bed Rest to Astronauts, American Society for Bone and Mineral Research, 25(5): 1049-1057; the contents of which are expressly incorporated by reference herein. For example, the effect of HMOs on NF-κB by simulated microgravity in differentiated osteoclasts can be evaluated using a high-aspect-ratio vessel (HARV) to provide an environment for the cells, similar to weightlessness as described by Zwart et al.

While this invention has been particularly shown and described with references to preferred embodiments thereof, it will be understood by those skilled in the art that various changes in form and details may be made therein without departing from the scope of the invention encompassed by the appended claims.

1. A method of treating a subject in a low gravity environment, comprising administering to said subject a composition comprising one or more human milk oligosaccharide, wherein such treatment prevents or slows the progression of dysbiosis, immune dysregulation, muscle wasting, metabolic disorders, cardiac atrophy or neurobehavioral abnormalities associated with such low gravity environment.

2. The method of claim 1, wherein the low gravity environment is a low planetary orbit, an interplanetary voyage or inhabiting a planet or moon with gravity less than 1 G.

3. The method of claim 2, wherein the low planetary orbit is selected from low Earth orbit, low Moon orbit or low Mars orbit.

4. The method of claim 1, wherein the composition is not a mammalian milk.

5. The method of claim 1, wherein the composition is not derived from mammalian milk.

6. The method of claim 1, wherein the composition comprises 10% or more, 20% or more, 30% or more, 40% or more, or 50% or more by mass one or more human milk oligosaccharides.

7. The method of claim 1, wherein the one or more human milk oligosaccharide is selected from lacto-N-tetraose (LNT), lacto-N-neotetraose (LNnT), lacto-N-hexaose (LNH), lacto-N-neohexaose (LNnH), 2′fucosyllactose (2′FL), 3′fucosyllacose (3′FL), lacto-difucotetraose (LDFT), lacto-N-fucopenaose (LNFP II/III), lactose-N-fucopentaose I (LNFP I), lacto-N-difuco-hexaose I (LNDFH I), lacto-N-difuco-hexaose II (LNDFH II), difucosyl-para-lacto-N-neohexaose (DFpLNnH), difucosyllacto-N-hexaose c (DFLNH c), 3′sialyllactose (3′SL), 6′ sialyllactose (6′SL), LS-tetrasaccharide a (LSTa), LS-tetrasaccharide b (LST b), LS-tetrasaccharide c (LST c), 3′-sialyl-N-acetyllactosamine (3′ SLN), 6′-sialyl-N-acetyllactosamine (6′ SLN), or disialyllacto-N-tetraose (DSLNT).

8. The method of claim 1, wherein the one or more human milk oligosaccharide selected from 2′FL, 3′FL, 3′SL, 6′SL, LNT, or LNnT.

9. The method of claim 1, wherein the composition comprising one or more human milk oligosaccharides contains a mixture of two, three, four or five human milk oligosaccharides.

10. The method of claim 9, wherein the composition comprises a mixture selected from:
i. 2′FL and LNT;
ii. 2′FL and LNnT;
iii. 2′FL, 3′FL, 3′SL, 6′SL and LNT;
iv. 3′SL and 6′SL; or
v. 6′ SL and LNT.

11. The method of claim 9, wherein the composition comprises a mixture of one neutral core and one neutral fucosylated human milk oligosaccharide.

12. The method of claim 9, wherein the composition comprises a mixture of one neutral and one acidic human milk oligosaccharide.

13. The method of claim 9, wherein the composition comprises a mixture of one neutral fucosylated and one acidic human milk oligosaccharide.

14. The method of claim 1, wherein the subject has exhibited signs or symptoms of one or more of the following disorders while in said low gravity environment: dysbiosis, immune dysregulation, muscle wasting, metabolic disorders, cardiac atrophy or neurobehavioral abnormalities associated with such low gravity environment.

15. The method of claim 14, wherein said dysbiosis in said subject is determined by fecal microbiome analysis.

16. The method of claim 15, wherein the fecal microbiome analysis is performed by 16S ribosomal RNA sequencing.

17. The method of claim 15, wherein the subject's fecal microbiome contains less Bifidobacterium and Faecalibacterium compared to the subject's microbiome while on Earth.

18. The method of claim 17, wherein the subject's fecal microbiome contains less F. prausnitzii compared to the subject's microbiome while on Earth.

19. The method of claim 14, wherein the subject in said low gravity environment has an increased level of salivary cortisol, plasma IL-6, plasma TNF-α, plasma c-reactive protein, plasma MCP-1, or plasma MIP1-α compared to levels in said subject on Earth.

20. A method of reducing signs or symptoms of a subject in a low gravity environment, comprising administering to said subject a composition comprising one or more human milk oligosaccharide, wherein said subject exhibits signs or symptoms of a disorder selected from irritable bowel syndrome, inflammatory bowel disease, osteoporosis, frailty, skin hypersensitivity, allergic reactions, viral infection, sarcopenia or inflammaging.

21. The method of claim 20, wherein the low gravity environment is a low planetary orbit, an interplanetary voyage or inhabiting a planet or moon with gravity less than 1 G.

22. The method of claim 21, wherein the low planetary orbit is selected from low Earth orbit, low Moon orbit or low Mars orbit.

23. The method of claim 20, wherein the composition is not a mammalian milk.

24. The method of claim 20, wherein the composition is not derived from mammalian milk.

25. The method of claim 20, wherein the composition comprises 10% or more, 20% or more, 30% or more, 40% or more, or 50% or more by mass one or more human milk oligosaccharide.

26. The method of claim 20, wherein the one or more human milk oligosaccharide is selected from lacto-N-tetraose (LNT), lacto-N-neotetraose (LNnT), lacto-N-hexaose (LNH), lacto-N-neohexaose (LNnH), 2′fucosyllactose (2′FL), 3′fucosyllacose (3′FL), lacto-difucotetraose (LDFT), lacto-N-fucopenaose (LNFP II/III), lactose-N-fucopentaose I (LNFP I), lacto-N-difuco-hexaose I (LNDFH I), lacto-N-difuco-hexaose II (LNDFH II), difucosyl-para-lacto-N-neohexaose (DFpLNnH), difucosyllacto-N-hexaose c (DFLNH c), 3′ sialyllactose (3′SL), 6′ sialyllactose (6′SL), LS-tetrasaccharide a (LSTa), LS-tetrasaccharide b (LST b), LS-tetrasaccharide c (LST c), 3′-sialyl-N-acetyllactosamine (3′ SLN), 6′-sialyl-N-acetyllactosamine (6′ SLN), or disialyllacto-N-tetraose (DSLNT).

27. The method of claim 20, wherein the one or more human milk oligosaccharide selected from 2′FL, 3′FL, 3′SL, 6′SL, LNT, or LNnT.

28. The method of claim 20, wherein the composition comprising one or more human milk oligosaccharides contains a mixture of two, three, four or five human milk oligosaccharides.

29. The method of claim 28, wherein the composition comprises a mixture selected from:
i. 2′FL and LNT;
ii. 2′FL and LNnT;
iii. 2′FL, 3′FL, 3′SL, 6′SL and LNT;
iv. 3′SL and 6′SL; or
v. 6′ SL and LNT.

30. The method of claim 28, wherein the composition comprises a mixture of one neutral core and one neutral fucosylated human milk oligosaccharide.

31. The method of claim 28, wherein the composition comprises a mixture of one neutral and one acidic human milk oligosaccharide.

32. The method of claim 20, wherein subject in said low gravity environment has an increased level of salivary cortisol, plasma IL-6, plasma TNF-α, plasma c-reactive protein, plasma MCP-1, plasma MIP1-α or fecal calprotectin compared to levels in said subject on Earth.

33. The method of claim 1, wherein the composition is in the form of a powder, incorporated into food, incorporated into supplements, or incorporated into water supply.
